(19) United States
(12) Patent Application Publication
(10) Pub. No.: US 2005/0277148 A1
(43) Pub. Date: Dec. 15, 2005

(54) Method for preparing correlation diagram or multiple alignment among nucleic acid sequences and program thereof

(75) Inventor: Shigeru Yatsuzuka, Tokyo (JP)

Correspondence Address: Reed Smith LLP, Suite 1400, 3110 Fairview Park Drive, Falls Church, VA, 22042-4503, US

(73) Assignee: Hitachi Software Engineering Co., Ltd.

(21) Appl. No.: 11/147,450

(22) Filed: Jun. 8, 2005

(30) Foreign Application Priority Data
Jun. 15, 2004 (JP) 177319/2004

(51) Int. Cl. C12Q 1/68; G06F 19/00; G01N 33/48; G01N 33/50

(52) U.S. Cl. 435/6; 702/20

(57) ABSTRACT

Means is provided by which correlation analysis among a plurality of nucleic acid sequences can be conducted in a high-speed manner on the basis of the considerations of a complementary strand of an analysis object sequence and highly accurate results can be obtained. Before conducting a correlation analysis, the directions of nucleic acid sequences, which are analysis objects, are determined, and correlation analysis becomes possible using input sequences whose directions have been determined.

### CLAIM OF PRIORITY

The present application claims priority from Japanese application JP 2004-177319 filed on Jun. 15, 2004, the content of which is hereby incorporated by reference into this application.

### BACKGROUND OF THE INVENTION

1. Field of the Invention

The present invention relates to a method for preparing a correlation diagram or a multiple alignment among nucleic acid sequences by conducting a correlation analysis among a plurality of nucleic acid sequences.

2. Background Art

In general, nucleic acid has two polynucleotide strands arranged in parallel via hydrogen bonding between bases and the polynucleotide strands twist with respect to each other to form a double helix structure. The bonding between the bases is based on hydrogen bonding between adenine (A) and thymine (T), and guanine (G) and cytosine (C) in a complementary manner, so that no other combination takes place. A polynucleotide strand bonded to a certain polynucleotide strand in a complementary manner is referred to as a complementary strand of the polynucleotide strand.

Conventionally, ClustalW (1994-), a program made by J. Thompson and T. Gibson, has been used as a method for conducting correlation analysis among biopolymers including nucleic acid. A calculation method used in the program is described in ClustalW Thompson JD, Higgins DG, Gibson TJ (Nucleic Acid Res. 1994 Nov: 4673-80). ClustalW analyzes genealogical relationships in evolution among different biopolymers and prepares a multiple alignment thereof.

Non-patent Document: Nucleic Acid Res. 1994 Nov: 4673-80

### SUMMARY OF THE INVENTION

The conventional correlation analysis, however, has the following problems.

1. In a case where the direction of a nucleic acid sequence (5′→3′ (+direction) or 3′→5′ (− direction)), which is a calculation object, is uncertain, significant results cannot be obtained from an analysis in many cases (the problem of the accuracy of analysis results).

As shown in FIG. 9, in a nucleic acid sequence, the head of the sequence is referred to as 5′ and the end of the sequence is referred to as 3′. The 5′→3′ direction is referred to as a + direction and the 3′→5′ direction is referred to as a − direction. When the nucleic acid sequence is decoded using a device such as a sequencer, the double strand of the nucleic acid sequence cannot be decoded in a simultaneous manner, so that polynucleotide strands 901 and 903 are decoded one by one. Also, the direction of decoding is always constant (when the strand is disposed in the upper position and a base 902 is disposed in the lower position, the strand is decoded from left). Thus, when a certain polynucleotide strand 901 is decoded in the + direction, the complementary strand 903 thereof is necessarily decoded in the − direction.

2. One of the methods to resolve the aforementioned problem 1 includes a method where the sequences of complementary strands of all nucleic acid sequences, which are objects of calculation, are prepared and these sequences are added to calculation objects. However, in this case, the number of nucleic acid sequences as the calculation objects is doubled and the amount of calculation time is approximately quadrupled (the problem of calculation time).

3. Further, in method 2, a half of sequences in analysis results are not significant relative to the results, so that result display becomes confusing (the problem of result display).

It is an object of the present invention to provide a method for conducting correlation analysis among a plurality of nucleic acid sequences in a high-speed manner on the basis of the considerations of a complementary strand of an analysis object sequence, and for deriving results of high accuracy.

In order to achieve the aforementioned object, in the present invention, upon conducting correlation analysis among a plurality of nucleic acid sequences, either an original sequence or a complementary strand sequence thereof is selected as an input so as to have more significant results, and a correlation diagram or a multiple alignment among nucleic acid sequences is prepared. In other words, a homology search is conducted among one particular sequence (hereafter referred to as a query) selected arbitrarily from nucleic acid sequences that are analysis objects and all the rest sequences of the analysis objects. On the basis of results thereof, which of an original sequence and a complementary strand sequence will make more significant analysis results is determined in each sequence, and the sequence thereof is selected as the analysis object. Then, correlation analysis is conducted among the sequences selected as the analysis objects. The method of the present invention can be performed by loading a program into a computer.

By selecting the direction of an analysis object sequence, the accuracy of analysis results can be improved, and the problem of calculation time can also be resolved, since the number of object sequences is not increased. Further, all the sequences displayed in analysis results include only those sequences that are significant for the results.

According to the present invention, by determining the directions of input sequences, correlation analysis among nucleic acid sequences, which has required huge amount of time and resulted in low accuracy, can be conducted in a high-speed manner and in high accuracy.

### BRIEF DESCRIPTION OF THE DRAWINGS

FIG. 1 shows a system configuration diagram.

FIG. 2 shows a system configuration diagram.

FIG. 3 shows an example of a dendrogram.

FIG. 4 shows an example of a multiple alignment.

FIG. 5 shows a procedure of sequence correlation analysis on the basis of the consideration of a complementary strand.

FIG. 6 shows an illustration of the determination of the directions of input sequences.

FIG. 7 shows an example of a user interface (main dialog) upon introducing nucleic acid sequences.

FIG. 8 shows a procedure of the use of a user interface upon introducing nucleic acid sequences.

FIG. 9 shows an illustration of the directions of nucleic acid sequences and decoding directions using a device.

### DESCRIPTION OF THE PREFERRED EMBODIMENTS

In the following, embodiments of the present invention are described concretely with reference to the drawings.

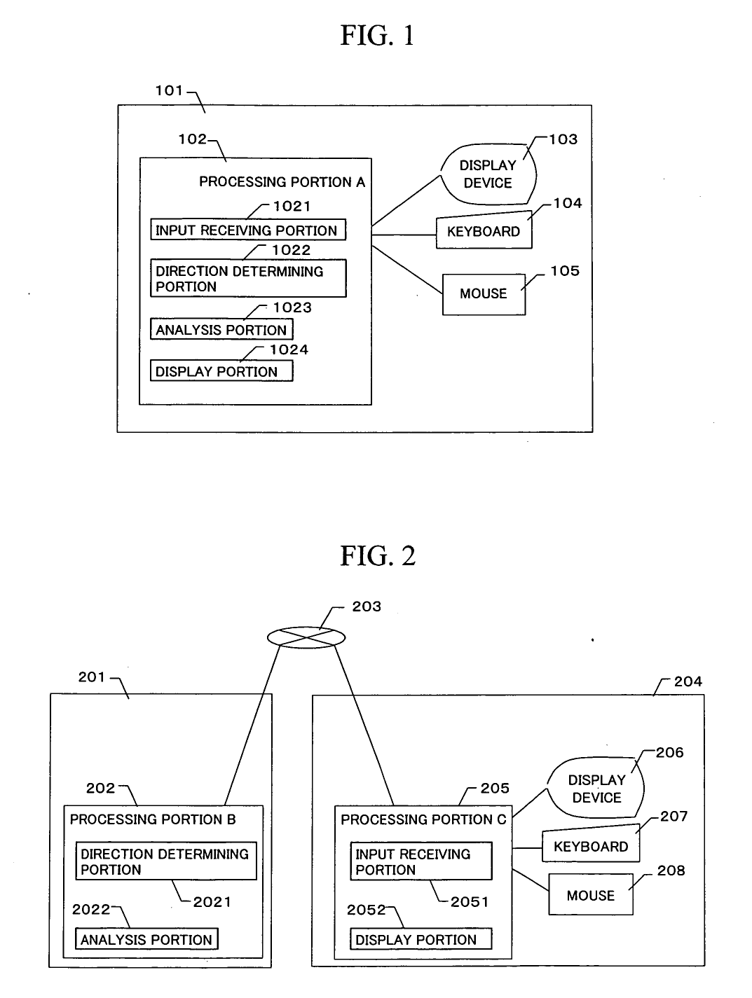

FIG. 1 shows a block diagram indicating an example of the configuration of a system (stand-alone type) for preparing a correlation diagram or a multiple alignment among nucleic acid sequences according to the present invention. As shown in FIG. 1, the present system (stand-alone type) is realized using a central processing unit 101. The present central processing unit 101 comprises a processing portion A102, a display device 103, a keyboard 104, and a mouse 105. The processing portion A102 comprises an input receiving portion 1021 for receiving input of sequences, a direction determining portion 1022 for determining the directions of input sequences, an analysis portion 1023 for conducting correlation analysis among sequences, and a display portion 1024 for performing a result display.

A user inputs an arbitrary nucleic acid sequence into the central processing unit 101 using the keyboard 104 or the mouse 105. The central processing unit 101 selects the directions of input sequences that make analysis results more significant, using the inputted nucleic acid sequence. Then, the central processing unit 101 conducts correlation analysis among these nucleic acid sequences and draws a correlation diagram or a multiple alignment among the nucleic acid sequences on the display device 103 on the basis of results thereof.

FIG. 2 shows another example of the configuration of a system (client/server type) for preparing a correlation diagram or a multiple alignment among nucleic acid sequences according to the present invention. As shown in FIG. 2, the present system (client/server type) is realized using a device 201 (server) for preparing a correlation diagram or a multiple alignment among nucleic acid sequences, a data input and output processing device (client) 204, and a communication channel 203. The device 201 for preparing a correlation diagram or a multiple alignment among nucleic acid sequences comprises a processing portion B202 for performing the calculation of the directions of the input nucleic acid sequences and a multiple alignment process. The processing portion B202 comprises a direction determining portion 2021 for determining the directions of the input sequences and an analysis portion 2022 for conducting correlation analysis among sequences. The data input and output processing device 204 comprises a processing portion C205 for performing input and output processes regarding data, a display device 206, a keyboard 207, and a mouse 208. The processing portion C205 comprises an input receiving portion 2051 for receiving input of sequences and a display portion 2052 for performing a result display.

A user inputs an arbitrary nucleic acid sequence into the data input and output processing device 204 using the keyboard 207 or the mouse 208. The data input and output processing device 204 transmits the inputted sequence to the device 201 for preparing a correlation diagram or a multiple alignment among nucleic acid sequences through the communication channel 203. The device 201 for preparing a correlation diagram or a multiple alignment among nucleic acid sequences conducts correlation analysis among nucleic acid sequences using the transmitted nucleic acid sequence, and transmits results thereof to the data input and output processing device 204 through the communication channel 203. The data input and output processing device 204 draws a correlation diagram or a multiple alignment among nucleic acid sequences on the display device 206 on the basis of the transmitted analysis results.

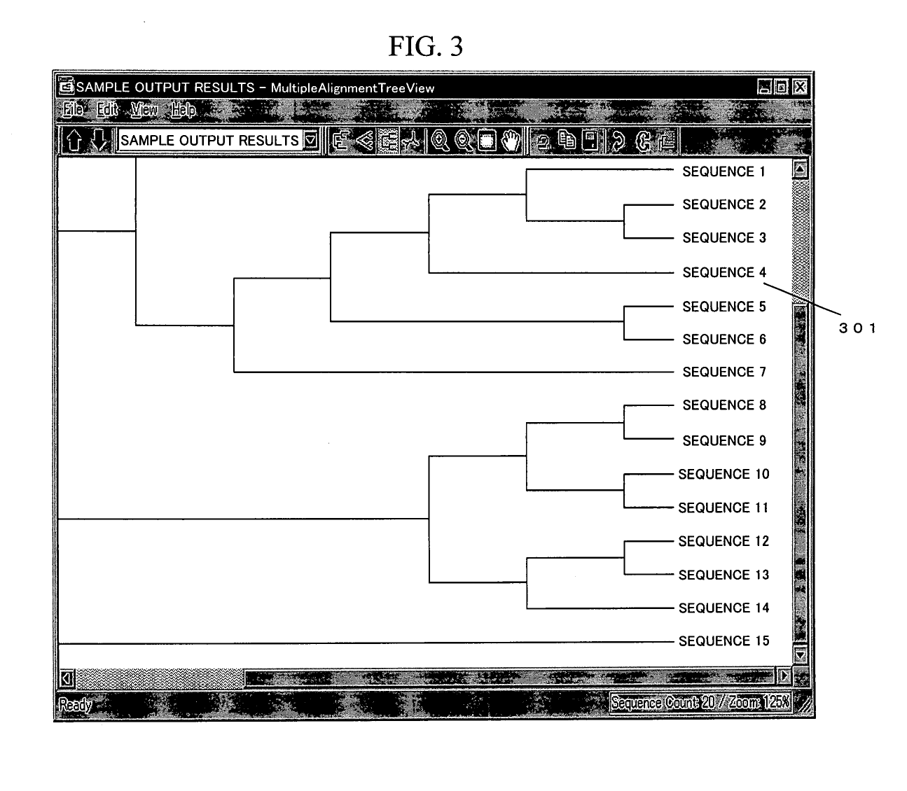

FIG. 3 shows an example of a dendrogram indicating a correlation among nucleic acid sequences displayed on the display device 103 or the display device 206. The dendrogram represents an evolutionary lineage among the nucleic acid sequences. Character strings 301 at the right end of the dendrogram represent sequence names of each sequence.

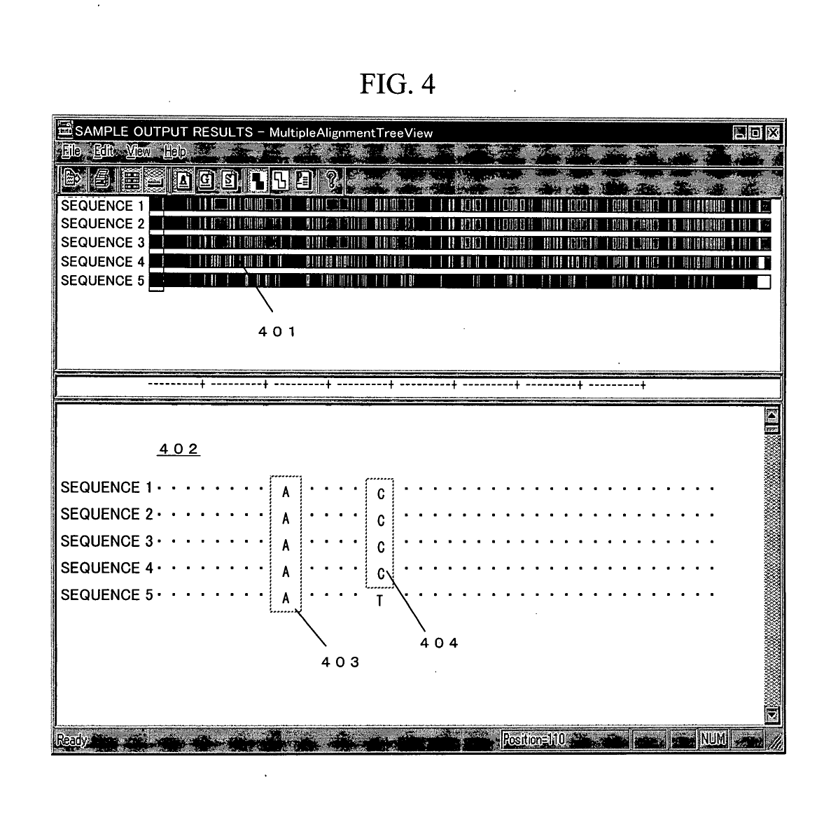

FIG. 4 shows an example of a multiple alignment (a plurality of sequences are arranged and displayed for ease of understanding of correspondence or noncorrespondence among the sequences) among nucleic acid sequences displayed on the display device 103 or the display device 206. The upper portion of a screen is allocated to a schematic view 401 of a multiple alignment, displaying the entire length of an alignment sequence. The lower portion of the screen is allocated to an alignment sequence 402. In the alignment sequence 402, it is possible to distinguish a portion 403 corresponding in all the sequences and a portion 404 having a certain level or more of a concordance rate in the sequences using different colors.

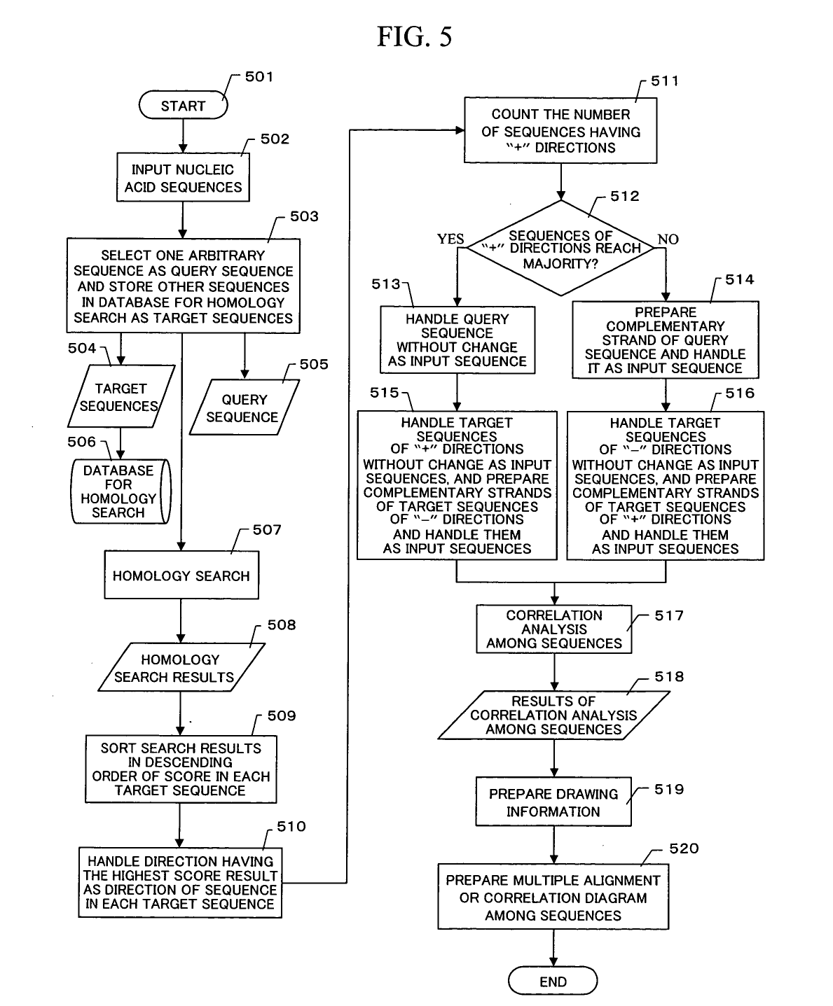

FIG. 5 shows a diagram describing the details of the preparation process of a correlation diagram or a multiple alignment among nucleic acid sequences in the systems for preparing a correlation diagram or a multiple alignment among nucleic acid sequences described in FIGS. 1 and 2. In this case, a homology search among nucleic acid sequences employs BLAST (Altschul, S. F., Madden, T. L., Schaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402.) or SSEARCH (D. J. Lipman, W. R. Person: Rapid and sensitive protein similarity searches, Science, 227, 1435-1441 (1985)), for example, as a program for searching for homologous sequences including a complementary strand. Correlation analysis among nucleic acid sequences employs ClustalW.

When the process is initiated (501), inputted sequences are read (502). Among the input sequences, one arbitrary sequence is handled as a query sequence 505, and the other sequences are handled as target sequences 504 (503). The target sequences 504 are stored in a database 506 for homology search.

Next, a homology search is conducted (507) among the query sequence 505 and the sequences in the database 506 for homology search. Search results 508 are sorted (509) in descending order of search score value in each target sequence. A direction of a nucleic acid sequence that indicates the highest score value in each target sequence of the results is handled as the direction of the sequence (510).

After the directions of the target sequences are determined, the number of sequences having “+” directions is counted (511). In a case where the sequences of “+” directions reach a majority, the query sequence is handled without change as an input sequence (513) for correlation analysis among sequences, the target sequences of “+” directions are handled without change as input sequences for correlation analysis among sequences, and complementary strands of the target sequences of “−” directions are prepared and handled as input sequences (515) for correlation analysis among sequences. In a case where the sequences of “+” directions do not reach a majority, a complementary strand of the query sequence is prepared and handled as an input sequence (514) for correlation analysis among sequences, the target sequences of “−” directions are handled without change as input sequences for correlation analysis among sequences, and complementary strands of the target sequences of “+” directions are prepared and handled as input sequences (516) for correlation analysis among sequences.

After the input sequences for correlation analysis among sequences are decided in this manner, the correlation analysis among sequences is conducted (517) and analysis results 518 are outputted. When the analysis results are outputted, information for drawing a correlation diagram or a multiple alignment among sequences is prepared (519), and the correlation diagram or the multiple alignment among sequences is drawn on a display device (520).

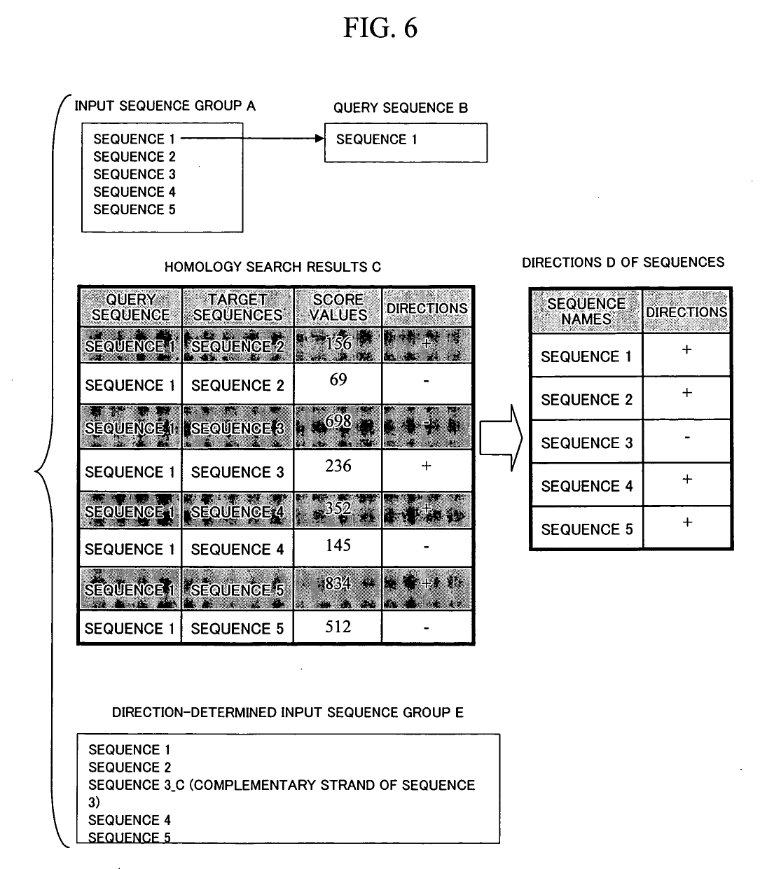

FIG. 6 shows a diagram describing the details of the determination process of the directions of the input sequences described in FIG. 5. First, an arbitrary sequence “sequence 1” is selected from an input sequence group A, and the sequence is handled as a query sequence B. Next, a homology search is conducted among the query sequence B and other sequences of the input sequence group A, and then research results C are obtained. In the search results C, by selecting an item that maximizes a score value in each target sequence, the direction thereof is obtained, and the directions D of the sequences are calculated. In this case, three sequences among four target sequences have “+” directions, so that the direction of the query sequence B is handled as “+” and the query sequence B is inserted into a direction-determined input sequence group E without change. Also, the direction of “sequence 3” of the target sequences is “−”, so that a complementary strand sequence “sequence 3_C” of the sequence is prepared and inserted into the direction-determined input sequence group E. Other target sequences are inserted into the direction-determined input sequence group E without change.

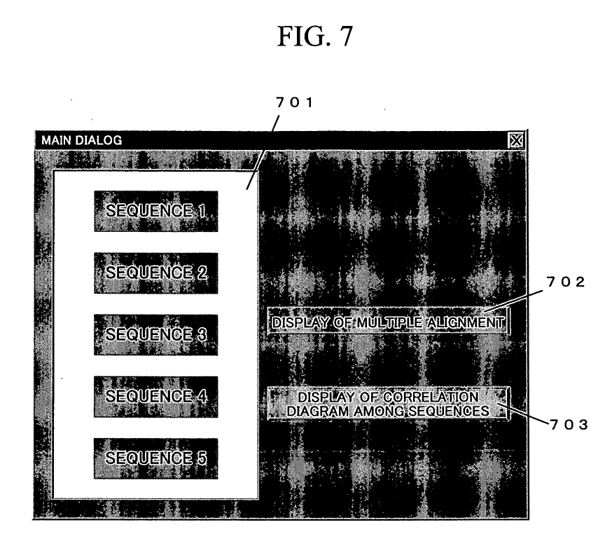

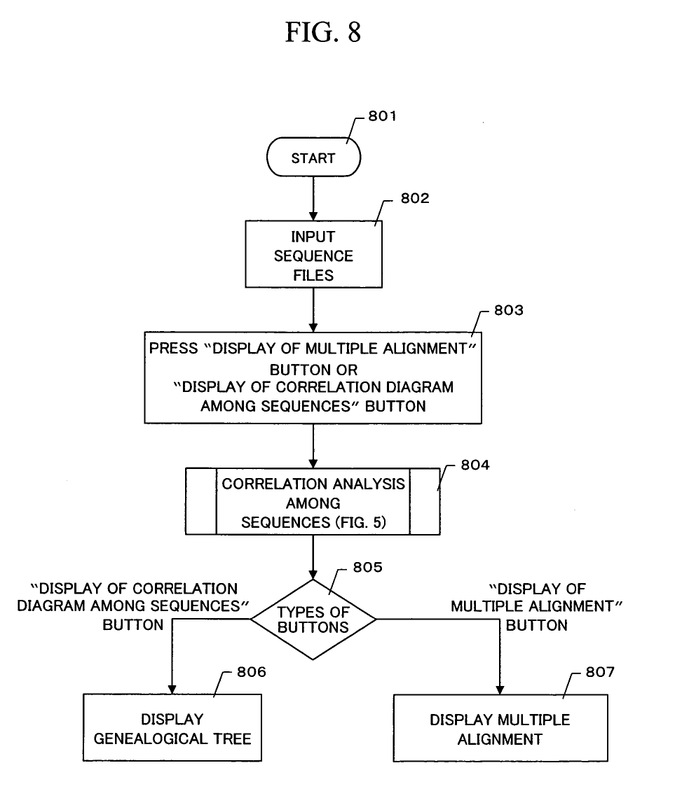

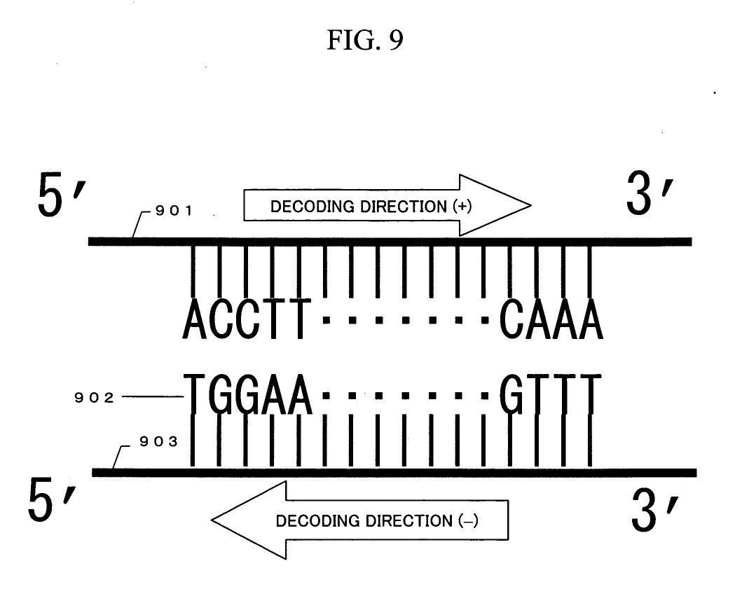

FIG. 7 shows an example of a mainly used dialog among user interfaces upon introducing nucleic acid sequences for a process of preparing a correlation diagram or a multiple alignment among nucleic acid sequences in the systems for preparing a correlation diagram or a multiple alignment among nucleic acid sequences described in FIGS. 1 and 2. First, in a main dialog (FIG. 7), a user drags and drops sequence files to input them into a file window 701. Next, the user can display a multiple alignment (FIG. 4) by pressing a “display of a multiple alignment” button 702 or a dendrogram (FIG. 3) indicating a correlation among sequences by pressing a “display of a correlation diagram among sequences” button 703.

FIG. 8 shows a diagram to describe the details of a procedure of the use of the user interface, as described in FIG. 7, upon introducing nucleic acid sequences for a process of preparing a correlation diagram or a multiple alignment among nucleic acid sequences in the system using a profile database.

When the process is initiated (801), sequence file input through drag and drop from a user is received (802). After the file input is completed, when the “display of a multiple alignment” button or the “display of a correlation diagram among sequences” button is pressed (803), correlation analysis among sequences is conducted (804). When the analysis is completed, the types of the buttons pressed by the user are determined (805). If the “display of a multiple alignment” button has been pressed, a multiple alignment is displayed (807), and if the “display of a correlation diagram among sequences” button has been pressed, a genealogical tree is displayed (806).

1. A method for preparing a correlation diagram or a multiple alignment among a plurality of nucleic acid sequences using a processing device provided with a homology search processing portion and a correlation analysis processing portion, wherein
the processing device performs the steps of:
handling one nucleic acid sequence of a plurality of inputted nucleic acid sequences as a query sequence and all the rest nucleic acid sequences as target sequences, and conducting a homology search among the query sequence, the target sequences, and complementary strand sequences thereof;
determining, on the basis of results of the homology search, whether the inputted nucleic acid sequences are used as analysis object sequences without change or whether complementary strand sequences of the inputted nucleic acid sequences are used as analysis object sequences in each of the inputted nucleic acid sequences, and conducting a correlation analysis among a plurality of the determined analysis object sequences; and
preparing, on the basis of results of the correlation analysis, a correlation diagram or a multiple alignment among the plurality of the nucleic acid sequences.

2. The method according to claim 1, wherein
the processing device performs the steps of:
determining in each target sequence, when sequences having high score values in the homology search are classified into the inputted nucleic acid sequences and the complementary strand sequences thereof, which of the sequences is larger in number; and
conducting correlation analysis, wherein
if the inputted nucleic acid sequences are determined to be larger in number as a result of the determination, the query sequence is handled as an analysis object sequence without change, and regarding the target sequences, inputted sequences are handled as analysis object sequences without change if the score value of the inputted nucleic acid sequence is higher, and complementary strand sequences of the inputted nucleic acid sequences are handled as analysis object sequences if the score value of the complementary strand sequence is higher, or
if complementary strand sequences are determined to be larger in number as a result of the determination, a complementary strand sequence of the query sequence is handled as an analysis object sequence, and regarding the target sequences, complementary strand sequences of the inputted sequences are handled as analysis object sequences if the score value of the inputted nucleic acid sequence is higher, and the inputted nucleic acid sequences are handled as analysis object sequences without change if the score value of the complementary strand sequence is higher.

3. A program for enabling a computer to perform the steps of:
handling one nucleic acid sequence of a plurality of inputted nucleic acid sequences as a query sequence and all the rest nucleic acid sequences as target sequences, and conducting a homology search among the query sequence, the target sequences, and complementary strand sequences thereof;
determining, on the basis of results of the homology search, whether the inputted nucleic acid sequences are used as analysis object sequences without change or whether complementary strand sequences of the inputted nucleic acid sequences are used as analysis object sequences in each of the inputted nucleic acid sequences, and conducting a correlation analysis among a plurality of the determined analysis object sequences; and
preparing, on the basis of results of the correlation analysis, a correlation diagram or a multiple alignment among the plurality of the nucleic acid sequences.

4. The program according to claim 3, comprising the steps of:
determining in each target sequence, when sequences having high score values in the homology search are classified into the inputted nucleic acid sequences and the complementary strand sequences thereof, which of the sequences is larger in number; and
conducting correlation analysis, wherein
if the inputted nucleic acid sequences are determined to be larger in number as a result of the determination, the query sequence is handled as an analysis object sequence without change, and regarding the target sequences, inputted sequences are handled as analysis object sequences without change if the score value of the inputted nucleic acid sequence is higher, and complementary strand sequences of the inputted nucleic acid sequences are handled as analysis object sequences if the score value of the complementary strand sequence is higher, or
if complementary strand sequences are determined to be larger in number as a result of the determination, a complementary strand sequence of the query sequence is handled as an analysis object sequence, and regarding the target sequences, complementary strand sequences of the inputted sequences are handled as analysis object sequences if the score value of the inputted nucleic acid sequence is higher, and the inputted nucleic acid sequences are handled as analysis object sequences without change if the score value of the complementary strand sequence is higher.
